(19) United States
(12) Patent Application Publication
(10) Pub. No.: US 2003/0093835 A1
(43) Pub. Date: May 15, 2003

(54) Chimeric genes controlling flowering

(76) Inventors: Detlef Weigel, Solana Beach, CA (US); Ji Hoon Ahn, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 09/919,478

(22) Filed: Jul. 30, 2001

(51) Int. Cl. A01H 1/00; C07H 21/04; C12N 9/16; C12N 5/04; C12P 21/02

(52) U.S. Cl. 800/287; 435/196; 435/69.1; 435/419; 435/320.1; 536/23.2

(57) ABSTRACT

The regulation of flowering time in plants is dependent upon the presence or absence of protein products of several flowering genes. Phosphatidylethanolamine-binding proteins are one such group of proteins involved in determining flowering time. Ectopic expression of members of this family may either confer early flowering or late flowering to the plant. A chimeric protein containing regions of a late-flowering member of the PEBP family combined with regions of an early flowering member of the PEBP family is disclosed here. A variety of gene segments from the PEBP-encoding genes may be swapped based on the degree of modulation of flowering time that is desired. Plants transformed with the genes encoding these chimeric proteins have a more carefully modulated flowering time.

### BACKGROUND OF THE INVENTION

1. Field of the Invention

This invention relates to plant genetic engineering, and specifically to the modulation of flowering time in plants.

2. Description of the Related Art

Plants have three types of meristems in their above ground architecture: vegetative, inflorescence, and floral. Vegetative growth generally dominates the initial stages of plant development. The plant then switches to a reproductive phase, where inflorescences, flowers, and fruit develop. The flowering transition is controlled by several interacting pathways influenced by both endogenous factors and in response to environmental signals. The transition of shoot apical meristems from the vegetative phase to the flowering phase is essential for the survival of the plant in its particular environment. Crops often need to be able to finish their flowering cycle before environmental stresses such as drought or cold occur. Early flowering is desirable in some species, such as plants that have a short growing season. Alternatively, late flowering may be more desirable in crops that produce vegetative products rather than reproductive products. Some plant species may not flower for several years after germination, which may be undesirable for breeding or crop yields. Moving crops from one environment, where late flowering is advantageous, to regions where earlier flowering is preferred, creates a need for methodologies that allow flowering times to be altered.

There are two basic types of inflorescences that occur upon the switch from the vegetative phase to the flowering phase: indeterminate and determinate. Species with indeterminate growth have apical meristems that grow indefinitely, generating floral meristems as they grow. Determinate species have apical meristems that produce floral meristems having terminal flowers that result in the termination of apical growth. Subsequent flowering must occur from lower, axillary meristems. Genetic modifications that alter the determinacy/indeterminacy pattern may therefore greatly alter the plant architecture, in addition to altering the flowering time.

Several genes that affect flowering time have been described, including APETALA1 (AP1), ARABIDOPSIS THALIANA CENTRORADIALIS (ATC), CONSTANS (CO), CENTRORADIALIS (CEN), TERMINAL FLOWER1 (TFL1), FLORICAULA (FLO), FLOWERING LOCUS T (FT), LEAFY (LFY), SQUAMOSA (SQUA), FLOWERING LOCUS CA (FCA), SELF-PRUNING (SP) and TWIN SISTER OF FT (TSF) (Amaya et al., Plant Cell, 11:1405 (1999); Coen et al., Cell, 63:1311 (1990); Huijser et al., EMBO J., 11:1239 (1992); Kardailsky, et al., Science, 286:1962 (1999); Mandel et al., Nature, 377:522 (1995); Michaels et al., Plant Cell, 11:949 (1999); Mimida et al., Genes Cells, 6:327 (2001); Pnueli et al., Development, 125:1979 (1998); Weigel et al., Cell, 69:843 (1992)). Proteins encoded by these genes interact in signal transduction pathways to control the architecture of the reproductive phase of the plant, and may be induced by endogenous or environmental signals. Several of these genes encode transcription factors, while others encode a group of polypeptides having sequence homology to mammalian polypeptides termed phosphatidylethanolamine-binding proteins (PEBPs).

This PEBP group of polypeptides includes FT (SEQ ID NO: 3) and its homolog TSF, as well as TFL1 (SEQ ID NO: 4) and its homolog ATC from Arabidopsis, along with CEN (from snapdragon), and SP (from tomato). A phylogenetic tree (FIG. 1) indicates the relationships between the PEBP family members from Arabidopsis.

Various PEBP homologs have been ectopically expressed in plants. Overexpression of TFL1 inhibits flowering in Arabidopsis but not in tobacco, while the snapdragon homolog CEN inhibits flowering when overexpressed in tobacco and Arabidopsis (Amaya et al., Plant Cell, 11:1405 (1999)); Ratcliffe et al., Development, 125:1609 (1998)). In tomato, homozygous recessive mutants of SELF PRUNING (SP) have a determinate floral architecture phenotype. Ectopic expression of SP in these plants rescues the indeterminate floral phenotype (Pnueli et al., Development, 125:1979 (1998)). Overexpression of ATC (the TFL1 homolog) produces the same late-flowering phenotype as TFL1 overexpression when ectopically expressed in Arabidopsis (Mimida et al., Genes Cells, 6:327 (2001)). Although the FT protein is structurally related to the other plant PEBPs (FIG. 3A), ectopic expression of FT results in the promotion of flowering rather than the inhibition of flowering that occurs with ectopic expression of TFL1, SP, and CEN. Overexpression of the FT homolog TSF (GenBank accession no. AB027506) produces the same phenotype as FT overexpression in Arabidopsis.

Mammalian members of the PEBP protein group have been found to be kinase inhibitors which regulate the RAF/MEK/ERK signal transduction pathway (Yeung et al., Nature, 401:173 (1999)). Additionally, mammalian PEBP proteins are believed to be the precursor of a bioactive peptide, as the first 12 amino acids of the PEBP comprise a hippocampal neurostimulatory peptide (HCNP) that plays a role in hippocampal development (Tohdoh et al., Mol. Brain Res., 30:381 (1995)).

Examination of the crystal structure of CEN, one of the plant homologs of FT, TFL1, and SP, confirmed sequence homology data suggesting that these proteins are a subset of PEBP-like proteins. When the crystal structure of CEN was compared with mammalian PEBPs, a highly conserved binding pocket was found embedded within the PEBP structure, formed from amino acid residues #70, 86, 110, and 120 (hPEBP numbering). This ligand binding site may “handle” phosphoryl ligands, which may result in the inhibition of kinase cascades (Banfield et al., J. Mol. Biol., 297:1159 (2000)). The PEBPs might act as kinase antagonists by binding to phosphorylated substrates or to their intermediate phosphorylated forms. Furthermore, a relatively non-conserved external loop region occurs between amino acids 128 and 145 in the CEN protein (FIG. 3A). This external loop is predicted to mediate protein-protein interactions (Banfield, supra (2000)).

Unfortunately, there is no simple way to modulate flowering by genetic manipulation. Thus, what is needed in the art is a method for modulating the flowering time of plants.

### BRIEF DESCRIPTION OF THE DRAWINGS

FIG. 1 is a phylogenetic tree of the FT/TFL1 family from Arabidopsis. Both FT and TFL1 have close homologs. TSF (Twin Sister of FT, GenBank accession no. AB027506) is a homolog of FT; TSF overexpression produces the same phenotype as overexpression of FT. ATC (Arabidopsis thaliana Centroradialis) is a homolog of TFL1; ATC overexpression produces the same phenotype as TFL1 overexpression (Mimida et al., supra (2001)). Inactivation of TSF or ATC gene expression does not produce a phenotype. CEN from snapdragon (Antirrhinum majus) is included for reference.

FIG. 2 is a block diagram illustrating embodiments of the chimeras tested under the 35S promoter in plants. FT is an early-flowering PEBP, while TFL1 is a late-flowering PEBP. E/L indicates degrees of earliness/lateness. “EEE” is similar to 35S::FT, while “LLL” is similar to 35S::TFL1.

FIG. 3A is an alignment comparing the amino acid sequences in part of the fourth exon between FT, TFL1 and the TFL1 orthologs, CEN from snapdragon and SP from tomato. Residues identical in at least three sequences are boxed. The gray box indicates the region swapped in plasmid pJA1122. The external loop seen in the CEN structure and predicted to mediate protein-protein interactions is shown below (Banfield and Brady, J. Mol. Biol., 297:1159 (2000)).

FIG. 3B is an alignment of FT and TFL1 amino acid sequences. Exon boundaries are indicated. In addition, break points used for the fourth-exon chimeras are also indicated.

FIG. 4A is a diagram of the FT (SEQ ID NO: 1) and TFL1 (SEQ ID NO: 2) genes, indicating the positions of PCR primers used to make the chimeras.

FIG. 4B is a diagram of the FT and TFL1 genes. Arrows indicate primer positions used to make the first round of PCR reactions needed to generate pJA1058.

FIG. 4C is a diagram of the FT and TFL1 genes. Arrows indicate primer positions used to make the second round of PCR reactions to prepare pJA1058.

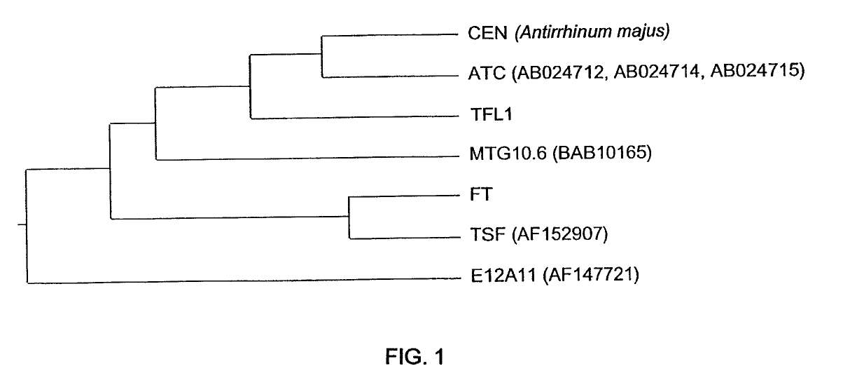

### SUMMARY OF THE INVENTION

One embodiment of the invention is a chimeric polypeptide that includes at least one region of an early-flowering phosphatidylethanolamine-binding protein, combined with at least one region of a late-flowering PEBP polypeptide, wherein the chimeric polypeptide has a different flowering time from that of a wild-type PEBP polypeptide.

Another embodiment of the invention is an isolated polynucleotide encoding a chimeric polypeptide. The chimeric polypeptide includes at least one region of an early-flowering phosphatidylethanolamine-binding protein, combined with at least one region of a late-flowering PEBP polypeptide, wherein the chimeric polypeptide has a different flowering time from that of a wild-type PEBP polypeptide.

Still another embodiment of the invention is an isolated polynucleotide selected from the group consisting of:

a) At least one region of SEQ ID NO: 1, or a sequence having substantial similarity thereto, combined with at least one region of SEQ ID NO: 2, or a sequence having substantial similarity thereto; and

b) At least one region of SEQ ID NO: 1, or a sequence having substantial similarity thereto, combined with at least one region of SEQ ID NO: 2, or a sequence having substantial similarity thereto, wherein T can also be U.

Yet another embodiment of the invention is a recombinant expression vector that includes an isolated polynucleotide selected from the group consisting of:

a) At least one region of SEQ ID NO: 1, or a sequence having substantial similarity thereto, combined with at least one region of SEQ ID NO: 2, or a sequence having substantial similarity thereto; and

b) At least one region of SEQ ID NO: 1, or a sequence having substantial similarity thereto, combined with at least one region of SEQ ID NO: 2, or a sequence having substantial similarity thereto, wherein T can also be U.

One other embodiment of the invention is a host cell containing a vector, wherein the vector has an isolated polynucleotide selected from the group consisting of:

a) At least one region of SEQ ID NO: 1, or a sequence having substantial similarity thereto, combined with at least one region of SEQ ID NO: 2, or a sequence having substantial similarity thereto; and

b) At least one region of SEQ ID NO: 1, or a sequence having substantial similarity thereto, combined with at least one region of SEQ ID NO: 2, or a sequence having substantial similarity thereto, wherein T can also be U.

An additional embodiment of the invention is an antibody which binds to a chimeric polypeptide that includes at least one region of an early-flowering phosphatidylethanolamine-binding protein, combined with at least one region of a late-flowering PEBP polypeptide, wherein the chimeric polypeptide has a different flowering time from that of a wild-type PEBP polypeptide. This embodiment also includes antibodies that bind to antigenic fragments of the chimeric polypeptide.

Still another embodiment is a genetically modified plant that includes at least one exogenous nucleic acid sequence comprising at least one region from an early PEBP-encoding nucleic acid sequence plus at least one region from a late PEBP-encoding nucleic acid sequence in its genome. Also within this embodiment are plant cells and plant tissue derived from the genetically modified plant.

Additionally, an embodiment of the invention is a seed which germinates into a plant that has at least one exogenous nucleic acid sequence comprising at least one region of an early PEBP-encoding nucleic acid sequence plus at least one region of a late PEBP-encoding nucleic acid sequence in its genome and characterized as having modulated flower development.

One other embodiment of the invention is a method for genetically modifying a plant cell such that a plant, produced from said cell, is characterized as having modulated flower development as compared with a wild-type plant. This method includes:

a) introducing at least the chimeric early PEBP/late PEBP-encoding polynucleotide of claim 1 into a plant cell to obtain a transformed plant cell; and

b) growing the transformed plant cell under conditions which permit expression of the early PEBP/late PEBP chimeric polypeptide, thereby producing a plant having modulated flower development.

Yet still another embodiment of the invention is a method of producing a genetically modified plant characterized as having early flower development, wherein the method includes:

contacting a plant cell with a vector containing a nucleic acid sequence comprising at least a structural gene encoding the chimeric early PEBP/late PEBP polypeptide, said gene operably associated with a promoter, to obtain a transformed plant cell;

producing a plant from said transformed plant cells; and

selecting a plant exhibiting said early flower development.

An additional embodiment of the invention is a method for modulating flower development in a plant cell. This method includes:

contacting plant cell with the vector of claim 33 to obtain a transformed plant cell;

growing the transformed plant cell under plant forming conditions; and

inducing early flower development in the plant under conditions and for a time sufficient to accelerate flower development.

One other embodiment of the invention is a method for identifying a compound that affects activity or expression of an early PEBP/late PEBP chimera that includes:

a) incubating components comprising the compound and the chimeric early PEBP/late PEBP polypeptide or a recombinant cell expressing the early PEBP/late PEBP chimeric polypeptide, under conditions that allow the components to interact; and

b) determining the effect of the compound on chimeric early PEBP/late PEBP activity or expression.

### DETAILED DESCRIPTION OF THE PREFERRED EMBODIMENT

Embodiments of the invention relate to the discovery that chimeric polypeptides comprised of regions of an early PEBP combined with regions of a late PEBP exhibit flowering characteristics that are different from either the early PEBP or the late PEBP. Additionally, these chimeras were found to exhibit different results when expressed in various plant species.

Embodiments of the invention are based on the discovery that chimeras of genes that control flowering often result in an altered flowering time when expressed in plants. In addition to creating transgenic plants that either flower very early or very late using overexpression of various chimeric PEBP-encoding genes, embodiments of the invention include methods for modulating the flowering system to create transgenic plants that have intermediate flowering time characteristics. Thus, embodiments of the invention provide the ability to further modulate flowering time by creating chimeras of proteins from regions of early-flowering members of the PEBP family of polypeptides combined with regions from the late-flowering members of the PEBP family of polypeptides.

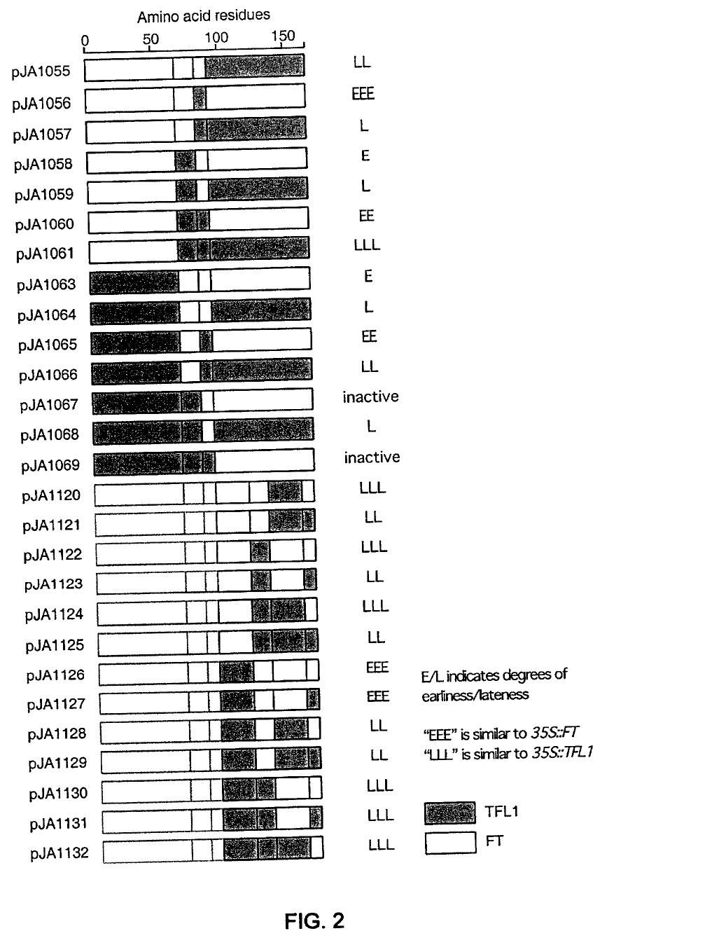

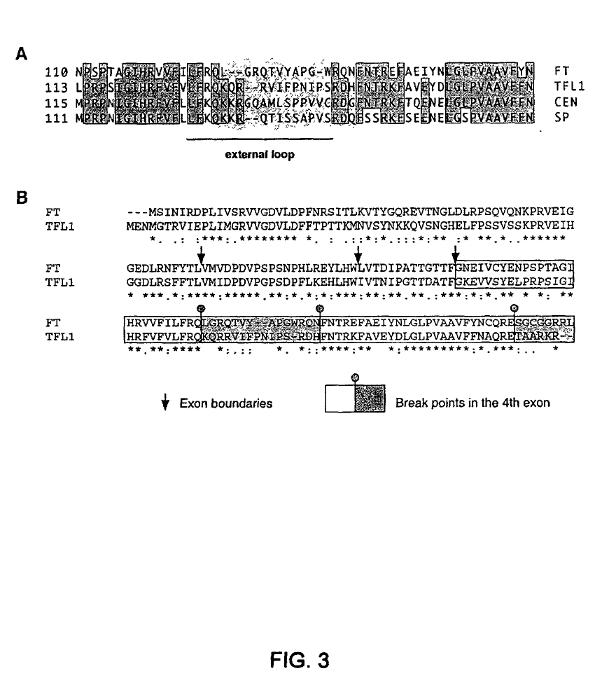

It was discovered that certain swapped segments of the late-flowering gene TFL1 (SEQ ID NO: 2) were especially effective in conferring modulated flowering time characteristics in plants with an early-flowering PEBP background (FIG. 2). For example, a DNA encoding 15 amino acid segment (amino acid #131-145) (SEQ ID NO: 39) swapped from the fourth exon of TFL1 (FIGS. 3A, 3B) was especially effective in conferring late-flowering characteristics when inserted into the homologous region of early-flowering PEBP genes.

One embodiment of the invention includes plants that overexpress early PEBP/late PEBP chimeras. Thus, embodiments of the invention provide isolated nucleotide sequences comprising, or derived from, the genes encoding early PEBPs combined with the genes encoding late PEBPs. Embodiments of the invention include not only the early PEBP/late PEBP chimeric sequences specifically, but also include splice variants of these sequences, allelic variants of these sequences, synonymous sequences, and homologous or orthologous variants of these sequences. Thus, for example, aspects of the invention provide genomic and cDNA chimeric sequences from an early PEBP gene combined with cDNA sequences from a late PEBP gene. Also included are allelic variants and homologous or orthologous sequences by providing methods by which such variants may be routinely obtained. The invention also provides for various nucleic acid constructs in which early PEBP/late PEBP chimeric sequences, either complete or subsets, are operably joined to exogenous sequences to form cloning vectors, expression vectors, fusion vectors, transgenic constructs, and the like.

Embodiments of the invention include plants, seedlings and methods of transferring the chimeric genes into plants in order to create plants having modulated flowering characteristics.

Manipulation of DNA Sequences

DNA sequences encoding early PEBP/late PEBP chimeric proteins can be expressed in vitro by DNA transfer into a suitable host cell. “Host cells” are the cells in which a vector is propagated and its DNA expressed. The term also includes any progeny or graft material, for example, of the subject host cell. It is understood that all progeny may not be identical to the parental cell since there may be mutations that occur during replication. However, such progenies are included when the term “host cell” is used. Methods of stable transfer, meaning that the foreign DNA is continuously maintained in the host, are known in the art.

In one step, the early PEBP/late PEBP chimeric polynucleotide sequences are inserted into a recombinant expression vector. The terms “recombinant expression vector” or “expression vector” refer to a plasmid, virus or other vehicle known in the art that has been manipulated by insertion or incorporation of the early PEBP/late PEBP chimeric genetic sequence. Such expression vectors typically contain a promoter sequence which facilitates the efficient transcription of the inserted early PEBP/late PEBP chimeric sequence. The expression vector typically contains an origin of replication, a promoter, as well as specific genes which allow phenotypic selection of the transformed cells.

Methods which are well known to those skilled in the art can be used to construct expression vectors containing the early PEBP/late PEBP chimeric coding sequence and appropriate transcriptional/translational control signals. These methods include in vitro recombinant DNA techniques, synthetic techniques, and in vivo recombination/genetic techniques.

A variety of host-expression vector systems may be utilized to express the chimeric early PEBP/late PEBP chimeric coding sequence. These include but are not limited to microorganisms such as bacteria transformed with recombinant bacteriophage DNA, plasmid DNA or cosmid DNA expression vectors containing the early PEBP/late PEBP chimeric coding sequence; yeast transformed with recombinant yeast expression vectors containing the early PEBP/late PEBP chimeric coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid or Ti plasmid-derived vectors) containing the early PEBP/late PEBP chimeric coding sequence; insect cell systems infected with recombinant virus expression vectors (e.g., baculovirus) containing the early PEBP/late PEBP chimeric coding sequence; or animal cell systems infected with recombinant virus expression vectors (e.g., retroviruses, adenovirus, vaccinia virus) containing the early PEBP/late PEBP chimeric coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al., Methods in Enzymology, 153:516-544 (1987)). Promoters produced by recombinant DNA or synthetic techniques may also be used to provide for transcription of the inserted chimeric early PEBP/late PEBP coding sequence.

Plant Transformation With Early/Late PEBP Chimeric Genes

In another embodiment, the invention provides a method for producing a genetically modified plant characterized as having modified flowering time characteristics as compared to a plant which has not been genetically modified (e.g., a wild-type plant). The method includes the steps of contacting a plant cell with at least one vector containing at least one nucleic acid sequence encoding an early PEBP/late PEBP chimera, wherein the nucleic acid sequence is operably associated with a promoter, to obtain a transformed plant cell; producing a plant from the transformed plant cell; and thereafter selecting a plant exhibiting modulated flowering time.

The term “genetic modification” as used herein refers to the introduction of one or more chimeric heterologous nucleic acid sequences, e.g., a chimeric early PEBP/late PEBP encoding sequence, into one or more plant cells, which can generate whole, sexually competent, viable plants. The term “genetically modified” as used herein refers to a plant which has been generated through the aforementioned process. Genetically modified plants are capable of self-pollinating or cross-pollinating with other plants of the same species so that the foreign gene, carried in the germ line, can be inserted into or bred into agriculturally useful plant varieties. The term “plant cell” as used herein refers to protoplasts, gamete producing cells, and cells which regenerate into whole plants. Accordingly, a seed comprising multiple plant cells capable of regenerating into a whole plant, is included in the definition of “plant cell”.

As used herein, the term “plant” refers to either a whole plant, a plant part, a plant cell, or a group of plant cells, such as plant tissue, for example. Plantlets are also included within the meaning of “plant”. Plants included in the invention are any plants amenable to transformation techniques, including angiosperms, gymnosperms, monocotyledons and dicotyledons.

Examples of monocotyledonous plants include, but are not limited to, asparagus, field and sweet corn, barley, wheat, rice, sorghum, onion, pearl millet, rye and oats. Examples of dicotyledonous plants include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals. Woody species include poplar, pine, sequoia, cedar, oak, etc.

In one embodiment, at least one nucleic acid sequence of an early PEBP/late PEBP chimeric polynucleotide is operably linked with a promoter. It may be desirable to introduce more than one copy of a chimeric early PEBP/late PEBP polynucleotide into a plant for enhanced expression. For example, multiple copies of the gene would have the effect of increasing production of the chimeric early PEBP/late PEBP in the plant.

Genetically modified plants are produced by contacting a plant cell with a vector including at least one nucleic acid sequence encoding a chimeric early PEBP/late PEBP. To be effective once introduced into plant cells, the chimeric early PEBP/late PEBP nucleic acid sequence should be operably associated with a promoter which is effective in the plant cells to cause transcription of the chimeric early PEBP/late PEBP chimeric gene. Additionally, a polyadenylation sequence or transcription control sequence, also recognized in plant cells may also be employed. It is preferred that the vector harboring the nucleic acid sequence to be inserted also contain one or more selectable marker genes so that the transformed cells can be selected from non-transformed cells in culture, as described herein.

The term “operably associated” refers to a functional linkage between a promoter sequence and a nucleic acid sequence regulated by the promoter. The operably linked promoter controls the expression of the nucleic acid sequence.

The expression of structural genes may be driven by a number of promoters. Although the endogenous, or native promoter of a structural gene of interest may be utilized for transcriptional regulation of the gene, the promoter may also be a foreign regulatory sequence. For plant expression vectors, suitable viral promoters include the 35S RNA and 19S RNA promoters of CaMV (Brisson, et al., Nature, 310:511 (1984); Odell, et al., Nature, 313:810 (1985)); the full-length transcript promoter from Figwort Mosaic Virus (FMV) (Gowda, et al., J. Cell Biochem., 13D: 301 (1989)) and the coat protein promoter to TMV (Takamatsu, et al., EMBO J., 6:307 (1987)). Alternatively, plant promoters such as the light-inducible promoter from the small subunit of ribulose-1,5-bisphosphate carboxylase (rbcS) (Coruzzi, et al., EMBO J., 3:1671 (1984)); Broglie, et al., Science, 224:838 (1984)); mannopine synthase promoter (Velten, et al., EMBO J., 3:2723 (1984)) nopaline synthase (NOS) and octopine synthase (OCS) promoters (carried on tumor-inducing plasmids of Agrobacterium tumefaciens) or heat shock promoters, e.g., soybean hsp17.5-E or hsp17.3-B (Gurley, et al., Mol. Cell. Biol., 6:559 (1986); Severin, et al., Plant Mol Biol., 15:827 (1990)) may be used.

Promoters useful in the invention include constitutive promoters, and inducible promoters, tissue-specific promoters, and inducible promoters. Useful promoters may be natural promoters as well as engineered promoters. The CaMV promoters are examples of constitutive promoters. To be most useful, an inducible promoter should 1) provide low expression in the absence of the inducer; 2) provide high expression in the presence of the inducer; 3) use an induction scheme that does not interfere with the normal physiology of the plant; and 4) have no effect on the expression of other genes. Examples of inducible promoters useful in plants include those induced by chemical means, such as the yeast metallothionein promoter which is activated by copper ions (Mett, et al., Proc. Natl. Acad. Sci., U.S.A., 90:4567 (1993)); In2-1 and In2-2 regulator sequences which are activated by substituted benzenesulfonamides, e.g., herbicide safeners (Hershey, et al., Plant Mol. Biol., 17:679 (1991)); and the GRE regulatory sequences which are induced by glucocorticoids (Schena, et al., Proc. Natl. Acad. Sci., U.S.A., 88:10421 (1991)). Tissue specific promoters may also be utilized in the present invention. An example of a tissue specific promoter is the promoter active in shoot meristems (Atanassova, et al., Plant J., 2:291 (1992)). Other tissue specific promoters useful in transgenic plants, including the cdc2a promoter and cyc07 promoter, will be known to those of skill in the art. (See for example, Ito, et al., Plant Mol. Biol., 24:863 (1994); Martinez, et al., Proc. Natl. Acad. Sci. USA, 89:7360 (1992); Medford, et al., Plant Cell, 3:359 (1991); Terada, et al., Plant Journal, 3:241 (1993); Wissenbach, et al., Plant Journal, 4:411 (1993)). Other promoters, both constitutive and inducible will be known to those of skill in the art.

The particular promoter selected should be capable of causing sufficient expression to result in the production of an effective amount of structural gene product, e.g., a chimeric early PEBP/late PEBP chimeric polypeptide, to cause modulated flowering time. The promoters used in the vector constructs of the present invention may be modified, if desired, to affect their control characteristics.

Optionally, a selectable marker may be associated with the nucleic acid sequence to be inserted. As used herein, the term “marker” refers to a gene encoding a trait or a phenotype which permits the selection of, or the screening for, a plant or plant cell containing the marker. Preferably, the marker gene is an antibiotic resistance gene whereby the appropriate antibiotic can be used to select for transformed cells from among cells that are not transformed. Examples of suitable selectable markers include adenosine deaminase, dihydrofolate reductase, hygromycin-B-phospho-transferase, thymidine kinase, xanthine-guanine phospho-ribosyltransferase and amino-glycoside 3′-O-phospho-transferase II (kanamycin, neomycin and G418 resistance). Other suitable markers will be known to those of skill in the art.

Vector(s) employed in the present invention for transformation of a plant cell include a nucleic acid sequence encoding a chimeric early PEBP/late PEBP polypeptide, operably associated with a promoter. To commence a transformation process in accordance with the present invention, a suitable vector is constructed and properly introduced into the plant cell. Details of the construction of vectors utilized herein are known to those skilled in the art of plant genetic engineering.

Early PEBP/late PEBP chimeric nucleic acid sequences can be introduced into plant cells using Ti plasmids of Agrobacterium tumefaciens, root-inducing (Ri) plasmids, and plant virus vectors. (For reviews of such techniques see, for example, Weissbach & Weissbach, Methods for Plant Molecular Biology, Academic Press, NY, Section VIII, pp. 421-463 (1988); and Grierson & Corey, Plant Molecular Biology, 2d Ed., Blackie, London, Ch. 7-9 (1988), and Horsch, et al., Science, 227:1229 (1985), both incorporated herein by reference). In addition to plant transformation vectors derived from the Ti or root-inducing (Ri) plasmids of Agrobacterium, alternative methods may involve, for example, the use of liposomes, electroporation, chemicals that increase free DNA uptake, transformation using viruses or pollen and the use of microprojection.

One of skill in the art can select an appropriate vector for introducing the chimeric early PEBP/late PEBP-encoding nucleic acid sequence in a relatively intact state. Thus, any vector which produces a plant carrying the introduced DNA sequence is sufficient. Even use of a naked piece of DNA is expected to confer the flowering time modulation properties described herein, though at low efficiency. The selection of the vector, or whether to use a vector, is typically guided by the method of transformation selected.

The transformation of plants may be carried out in essentially any of the various ways known to those skilled in the art of plant molecular biology (See, for example, Methods of Enzymology, Vol. 153 (1987), Wu and Grossman, Eds., Academic Press, incorporated herein by reference). As used herein, the term “transformation” means alteration of the genotype of a host plant by the introduction of a chimeric early PEBP/late PEBP nucleic acid sequence.

For example, a chimeric early PEBP/late PEBP nucleic acid sequence is introduced into a plant cell utilizing Agrobacterium tumefaciens containing the Ti plasmid, as mentioned briefly above. In using an A. tumefaciens culture as a transformation vehicle, it is most advantageous to use a non-oncogenic strain of Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The latter contains at least one border region of the T-DNA region of a wild-type Ti plasmid flanking the nucleic acid to be transferred. Binary Ti plasmid systems have been shown effective to transform plant cells (De Framond, Biotechnology, 1: 262 (1983); Hoekema, et al., Nature, 303:179 (1983)). Such a binary system is preferred because it does not require integration into the Ti plasmid of Agrobacterium, which is an older methodology.

Methods involving the use of Agrobacterium in transformation include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by the method of vacuum infiltration of plant tissue with a suspension of Agrobacterium harboring the T-DNA plasmid of interest, as described by Bechtold, et al. (C. R. Acad. Sci. Paris, 316:1194 (1993)).

One method of introducing chimeric early PEBP/late PEBP-encoding nucleic acid into plant cells is to infect such plant cells, an explant, a meristem or a seed, with transformed Agrobacterium tumefaciens as described above. Under appropriate conditions known in the art, the transformed plant cells are grown to form shoots, roots, and develop further into plants.

Alternatively, chimeric early PEBP/late PEBP-encoding nucleic acid sequences can be introduced into a plant cell using mechanical or chemical means. For example, the nucleic acid can be mechanically transferred into the plant cell by microinjection using a micropipette. Alternatively, the nucleic acid may be transferred into the plant cell by using polyethylene glycol which forms a precipitation complex with genetic material that is taken up by the cell.

Chimeric early PEBP/late PEBP nucleic acid sequences can also be introduced into plant cells by electroporation (Fromm, et al., Proc. Natl. Acad. Sci., U.S.A., 82:5824 (1985)), which is incorporated herein by reference). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing chimeric early PEBP/late PEBP nucleic acid sequences into a plant cell is high velocity ballistic penetration by small particles, wherein the nucleic acid to be introduced is contained either within the matrix of such particles, or on the surface thereof (Klein, et al., Nature 327:70 (1987)). Bombardment transformation methods are also described in Sanford, et al. (Techniques 3:3-16 (1991)) and Klein, et al. (Bio/Techniques, 10:286 (1992)). Although, typically only a single introduction of a new nucleic acid sequence is required, this method particularly provides for multiple introductions.

Cauliflower mosaic virus (CaMV) may also be used as a vector for introducing nucleic acid into plant cells (U.S. Pat. No. 4,407,956). CaMV viral DNA genome is inserted into a parent bacterial plasmid creating a recombinant DNA molecule which can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

As used herein, the term “contacting” refers to any means of introducing chimeric early PEBP/late PEBP into the plant cell, including chemical and physical means as described above. Preferably, contacting refers to introducing the nucleic acid or vector into plant cells (including an explant, a meristem or a seed), via Agrobacterium tumefaciens transformed with the chimeric early PEBP/late PEBP-encoding nucleic acid as described above.

Regeneration of Transformed Plants

Normally, a plant cell is regenerated to obtain a whole plant from the transformation process. The immediate product of the transformation is referred to as a “transgenote”. The term “growing” or “regeneration” as used herein means growing a whole plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for plant species such as corn and alfalfa. Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration. (see Methods in Enzymology, Vol. 118 and Klee, et al., Annual Review of Plant Physiology, 38:467 (1987)). Utilizing the leaf disk-transformation-regeneration method of Horsch, et al. (Science, 227:1229 (1985)), disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by utilizing cuttings or tissue culture techniques to produce multiple identical plants. Selection of desirable transgenotes is made and new varieties are obtained and propagated vegetatively for commercial use.

In seed propagated crops, the mature transgenic plants can be self crossed to produce a homozygous inbred plant. The resulting inbred plant produces seed containing the newly introduced foreign gene(s). These seeds can be grown to produce plants that would produce the selected phenotype, e.g. increased yield.

Parts obtained from regenerated plant, such as flowers, seeds, leaves, branches, roots, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

Genetically Modified Plants With Modulated Flowering Time

Plants exhibiting modified flowering time as compared with wild-type plants can be selected by visual observation. Embodiments of this invention include plants produced by the method of the invention, as well as plant tissue and seeds.

Yet another embodiment is a method for producing a genetically modified plant cell such that a plant produced from the cell has a modified flowering time as compared with a wild-type plant. The method includes contacting the plant cell with a chimeric early PEBP/late PEBP nucleic acid sequence to obtain a transformed plant cell; growing the transformed plant cell under plant forming conditions to obtain a whole plant.

“Flowering time” refers to the onset of the flowering phase of plant development. Several methods can be used to measure flowering time. In one method, the appearance of rosette leaves and cauline leaves of bolting plants are measured. Alternatively, the date of the formation of the first flower can be measured. “Flowering time” may also be described as the length of time in which a plant is in the flowering phase of its life cycle. The range of time when the plant exhibits the flowering phase may be measured and compared with that of wild-type plants. The flowering time may be sensitive to environmental conditions such as nutrient stress, cold stress, drought stress, or alterations in light quality or quantity. Therefore, conditions surrounding both the wild-type plant and the early PEBP/late PEBP-modified plant should be carefully controlled in order for an accurate comparison to be performed.

In another embodiment, the invention provides a method of producing a plant having modulated flowering time by contacting a susceptible plant with an agent which induces chimeric early PEBP/late PEBP gene expression in a transformed plant, wherein induction of the chimeric gene results in production of a plant having a modified flowering time as compared to a plant not contacted with the agent.

A “susceptible plant” refers to a plant that can be induced to utilize its chimeric early PEBP/late PEBP gene to achieve modified flowering time. The term “promoter inducing amount” refers to that amount of an agent necessary to elevate chimeric early PEBP/late PEBP gene expression above chimeric early PEBP/late PEBP expression in a plant cell not contacted with the agent. For example, a transcription factor or a chemical agent may be used to elevate gene expression from the native promoter of a PEBP gene, thus inducing the promoter and expression of the early PEBP/late PEBP chimeric gene.

The above disclosure generally describes the present invention. A more complete understanding can be obtained by reference to the following specific examples which are provided herein for purposes of illustration only and are not intended to limit the scope of the invention.

Chimeric Sequence Modification

Embodiments of the invention include functional early PEBP/late PEBP chimeric polypeptides, and functional fragments thereof. As used herein, the term “functional polypeptide” refers to a polypeptide which possesses biological function or activity which is identified through a defined functional assay and which is associated with a particular biologic, morphologic, or phenotypic alteration in the cell.

As used herein, the term “substantially pure” as used herein refers to polypeptides which are substantially free of other proteins, lipids, carbohydrates or other materials with which it is naturally associated. One skilled in the art can purify a polypeptide using standard techniques for protein purification. The purity of a polypeptide can also be determined by amino-terminal amino acid sequence analysis.

Isolation and purification of recombinantly expressed polypeptides, or fragments thereof, may be carried out by conventional means including preparative chromatography and immunological separations involving monoclonal or polyclonal antibodies.

Many modifications resulting in an early PEBP/late PEBP chimeric polypeptide primary amino acid sequence were found to result in plants having modulated flowering time. Deletion of one or more amino acids can also result in a modification of the structure of the resultant molecule without significantly altering its activity. This can lead to the development of a smaller active molecule, which could have broader utility. For example, it may be possible to remove amino or carboxyl terminal amino acids required for early PEBP/late PEBP chimeric polypeptide activity.

Early PEBP/late PEBP chimeric polypeptides include amino acid sequences substantially the same as the sequence set forth in SEQ ID NO: 41 including mutants that result in plants having modulated flowering time. The term “substantially the same” refers to amino acid sequences that have a similar amino acid sequence and retain the activity of early PEBP/late PEBP chimeric polypeptides as described herein. The early PEBP/late PEBP chimeric polypeptides of the invention can include conservative variations of the polypeptide sequence.

The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

Early PEBP/late PEBP chimeric proteins can be analyzed by standard SDS-PAGE and/or immunoprecipitation analysis and/or Western blot analysis, for example.

Embodiments of the invention also provide an isolated chimeric polynucleotide sequence encoding a chimeric polypeptide having the amino acid sequence of SEQ ID NO: 41. The term “isolated” as used herein includes polynucleotides substantially free of other nucleic acids, proteins, lipids, carbohydrates or other materials with which it is naturally associated. Polynucleotide sequences of the invention include DNA, cDNA and RNA sequences which encode early PEBP/late PEBP chimeras. It is understood that polynucleotides encoding all or varying portions of early PEBP/late PEBP chimeric proteins are included herein, as long as they encode a polypeptide which exhibits a modulation of flowering time. Such polynucleotides include naturally occurring, synthetic, and intentionally manipulated polynucleotides as well as splice variants. For example, portions of the mRNA sequence may be altered due to alternate RNA splicing patterns or the use of alternate promoters for RNA transcription.

Moreover, early PEBP/late PEBP chimeric polynucleotides include polynucleotides having alterations in the nucleic acid sequence which still encode a polypeptide having the ability to alter flowering time. Alterations in early PEBP/late PEBP chimeric nucleic acids include, but are not limited to, intragenic mutations (e.g., point mutation, nonsense (stop), antisense, splice site and frameshift) and heterozygous or homozygous deletions. Detection of such alterations can be done by standard methods known to those of skill in the art including sequence analysis, Southern blot analysis, PCR based analyses (e.g., multiplex PCR, sequence tagged sites (STSs)) and in situ hybridization. Embodiments of the invention also include anti-sense polynucleotide sequences.

The polynucleotides described herein include sequences that are degenerate as a result of the genetic code. There are 20 natural amino acids, most of which are specified by more than one codon. Therefore, all degenerate nucleotide sequences are included in the invention as long as the amino acid sequence of the early PEBP/late PEBP chimeric polypeptide encoded by such nucleotide sequences retains chimeric early PEBP/late PEBP activity. A “functional polynucleotide” denotes a polynucleotide which encodes a functional polypeptide as described herein. In addition, embodiments of the invention also include a polynucleotide encoding a polypeptide having the biological activity of an amino acid sequence of SEQ ID NO: 41 and having at least one epitope for an antibody immunoreactive with the early PEBP/late PEBP chimeric polypeptide.

Another aspect of the invention is polypeptides or fragments thereof which have at least about 70%, at least about 80%, at least about 85%, at least about 90%, at least about 95%, or more than about 95% homology to one of the polypeptides of SEQ ID NO: 3, and sequences substantially identical thereto, or a fragment comprising at least 5, 10, 15, 20, 25, 30, 35, 40, 50, 75, 100, or 150 consecutive amino acids thereof. Homology may be determined using any of the methods described herein which align the polypeptides or fragments being compared and determines the extent of amino acid identity or similarity between them. It will be appreciated that amino acid “homology” includes conservative amino acid substitutions such as those described above.

Also included in embodiments of the invention are nucleotide sequences that are greater than 70% homologous with the sequence of SEQ ID NO: 1, but still retain the ability to modify flowering time. Other embodiments of the invention include nucleotide sequences that are greater than 75%, 80%, 85%, 90% or 95% homologous with the sequence of SEQ ID NO: 1, but still retain the ability to modulate flowering time. As used herein, the terms “polynucleotides” and “nucleic acid sequences” refer to DNA, RNA and cDNA sequences.

Identification of Interacting Molecules

In another series of embodiments, the present invention provides methods for identifying proteins and other compounds which bind to, or otherwise directly interact with, the early PEBP/late PEBP. The proteins and compounds will include endogenous cellular components which interact with the early PEBP/late PEBP chimera in vivo and which, therefore, provide new targets for agricultural products, as well as recombinant, synthetic and otherwise exogenous compounds which may have early PEBP/late PEBP binding capacity. Alternatively, any of a variety of exogenous compounds, both naturally occurring and/or synthetic (e.g., libraries of small molecules or peptides), may be screened for early PEBP/late PEBP chimera binding capacity.

In each of these embodiments, an assay is conducted to detect binding between the early PEBP/late PEBP chimera and some other moiety. Binding may be detected by non-specific measures (e.g., transcription modulation, altered chromatin structure, peptide production or changes in the expression of other downstream genes which can be monitored by differential display, 2D gel electrophoresis, differential hybridization, or SAGE methods) or by direct measures such as immunoprecipitation, the Biomolecular Interaction Assay (BIAcore) or alteration of protein gel electrophoresis. The preferred methods involve variations on the following techniques: (1) direct extraction by affinity chromatography; (2) co-isolation of the early-flowering PEBP/late-flowering PEBP chimeric components and bound proteins or other compounds by immunoprecipitation; (3) BIAcore analysis; and (4) the yeast two-hybrid systems.

Embodiments of the present invention also provide for methods of identifying proteins, small molecules and other compounds capable of modulating the activity of an early PEBP/late PEBP chimeric protein. Using prokaryotic or eukaryotic cells, especially plant cells or whole plants, the transformed cells and plant models of the present invention, or cells obtained from plants bearing early PEBP/late PEBP chimeric genes, methods of identifying such compounds are provided on the basis of their ability to affect the expression of early PEBP/late PEBP chimeric sequences, the activity of early PEBP/late PEBP chimeric sequences, the activity of other early PEBP/late PEBP chimeric genes, the activity of proteins that interact with early PEBP/late PEBP chimeric proteins, the intracellular localization of the early PEBP/late PEBP chimeric protein, changes in transcriptional activity, or other biochemical, histological, or physiological markers which distinguish cells bearing chimeric early PEBP/late PEBP activity in plants.

A further embodiment of the present invention is a method of identifying a compound that affects the activity or expression of a chimeric early PEBP/late PEBP encoding gene by directly measuring changes in flowering time. A plant cell containing the chimeric early PEBP/late PEBP encoding gene is first treated with the molecule. The molecule may be exogenously applied or inserted into the cell. The molecule may also be a nucleic acid that is inserted into the cell in either a stable or transient fashion. After a variable incubation which would be determined by experiment, the change in flowering time may be measured either by counting the leaves produced before flowering commences, or by measuring the number of days from addition of the test molecule to the date of the onset of flowering.

In accordance with another aspect of the invention, these chimeric proteins can be used as starting points for rational chemical design to provide ligands or other types of small chemical molecules. Alternatively, small molecules or other compounds identified by the above-described screening assays may serve as “lead compounds” in design of modulators of flowering time in plants.

### EXAMPLE 1

### Generation of the Chimeric Genes by Swapping Exons

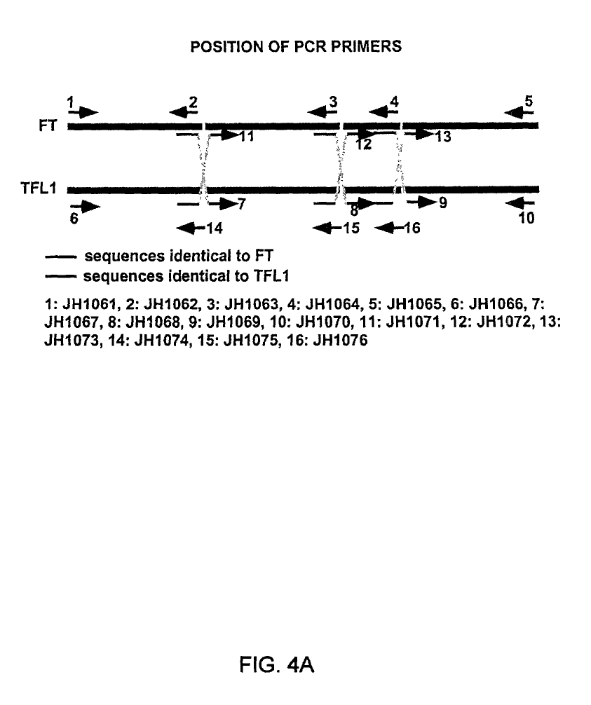

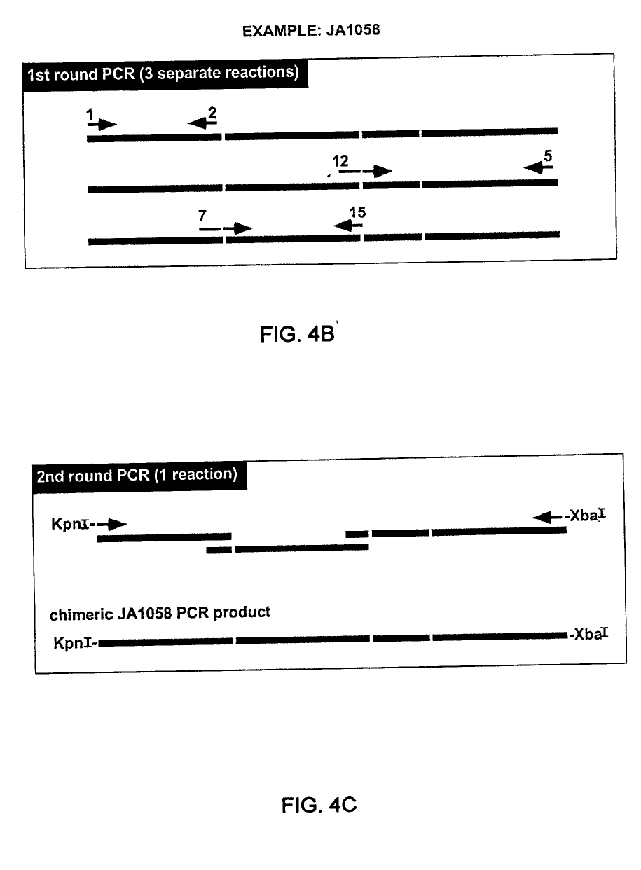

The chimeric genes containing regions of the early PEBP FT combined with regions of the late PEBP TFL1 (FIG. 2) were prepared by the following method. Synthetic oligonucleotide primers (JH1061-JH1092, listed below) flanking the exon boundaries (FIG. 3) were designed to allow the generation of the desired PCR fragments (FIG. 4A). The first half (about 20 nucleotides) of each oligonucleotide corresponds to the FT nucleotide sequence (SEQ ID NO: 1), the second half to the TFL1 nucleotide sequence (SEQ ID NO: 2). The annealing temperature of each half was designed to be between 57° C. and 61° C., while the annealing temperature of the entire oligonucleotide primer was designed to be between 64° C. and 80° C.

| | | |
|---|---|---|
| JH1061 | ATGTCTATAAATATAAGAGACCCTCTT | (SEQ ID NO:5) |
| | ATAGTAA | |
| JH1062 | CAAAGTATAGAAGTTCCTGAGGTCTT | (SEQ ID NO:6) |
| JH1063 | CAATGGAGATATTCTCGGAGGT | (SEQ ID NO:7) |
| JH1064 | CAAAGGTTGTTCCAGTTGTAGCA | (SEQ ID NO:8) |
| JH1065 | CTAAAGTCTTCTTCCTCCGCA | (SEQ ID NO:9) |
| JH1066 | ATGGAGAATATGGGAACTAGAG | (SEQ ID NO:10) |
| JH1067 | CCTCAGGAACTTCTATACTTTGGTGAT | (SEQ ID NO:11) |
| | GATAGACCCAGATGTTC | |
| JH1068 | TCACCTCCGAGAATATCTCCATTGGAT | (SEQ ID NO:12) |
| | CGTTACAAACATTCCCGG | |
| JH1069 | TGCTACAACTGGAACAACCTTTGGCAA | (SEQ ID NO:13) |
| | AGAGGTGCGTGAGCTATG | |
| JH1070 | CTAGCGTTTGCGTGCAGC | (SEQ ID NO:14) |
| JH1071 | GATCTCAGATCCTTCTTCACTTTGGTT | (SEQ ID NO:15) |
| | ATGGTGGATCCAGATGTTC | |
| JH1072 | CTTTCTAAAAGAACACCTGCACTGGTT | (SEQ ID NO:16) |
| | GGTGACTGATATCCCTGC | |
| JH1073 | GCACAACAGATGCTACGTTTGGCAATG | (SEQ ID NO:17) |
| | AGATTGTGTGTTACGAA | |
| JH1074 | CAAAGTGAAGAAGGATCTGAGATC | (SEQ ID NO:18) |
| JH1075 | CAGTGCAGGTGTTCTTTTAGAAAG | (SEQ ID NO:19) |
| JH1076 | CAAACGTAGCATCTGTTGTGC | (SEQ ID NO:20) |
| JH1089 | GAGGTACCATGTCTATAAATATAAGAG | (SEQ ID NO:21) |
| | ACCCTCTTATAGTAA | |
| JH1090 | GATCTAGACTAAAGTCTTCTTCCTCCG | (SEQ ID NO:22) |
| | CA | |
| JH1091 | AAGGTACCATGGAGAATATGGGAACTA | (SEQ ID NO:23) |
| | CA | |
| JH1092 | TCTCTAGACTAGCGTTTGCGTGCAGC | (SEQ ID NO:24) |

The first round of PCR amplification was carried out to generate appropriate pieces from FT or TFL1 cDNA at the annealing temperature of 55° C. The amplification process was performed according to the manufacturer's instructions (Stratagene) with the following exception: the Pfu enzyme (Lundberg et al., Gene, 108:1-6 (1991)) and the hot-start method (Bassam et al., Biotechniques, 14:30-4 (1993)) were used to minimize errors. Small fragments of less than 60 bp were directly synthesized as complementary oligonucleotides, which were subsequently annealed.

Following gel-purification of the desired fragments obtained from the first round of amplification, the appropriate combinations of fragments were combined and amplified with two oligonucleotide primers flanking the ends of the final chimeric constructs. The primers were designed to provide a synthetic KpnI restriction site before the translational start codon of the chimera and a synthetic XbaI restriction site after the translational stop codon. Therefore, all the chimeric genes generated contain only coding sequences between KpnI and XbaI sites.

The products were digested with KpnI and XbaI restriction enzymes and subcloned into pBluescript II KS+ (Stratagene) or pGEM3Zf(+) (Promega) after gel-purification. The sequences of the chimeric genes were then verified by DNA sequencing.

For example, to generate the clone pJA1058 (FIG. 2), which is the FT gene with a swapped second exon from the TFL1 gene, fragments (FIG. 4B) were amplified from the FT cDNA (Kardailsky et al., Science, 286:1962-1965 (1999)). Fragment 1 was amplified using primers JH1061 and JH1062 (described previously) and contained only FT sequence. Fragment 2 was amplified using primer JH1072 (SEQ ID NO: 16), a chimeric primer whose 5′ end consisted of TFL1 sequence, and JH1065 (SEQ ID NO: 9). The resulting product consisted mostly of an FT fragment linked to a short TFL1 sequence at the 5′ end. A third fragment was amplified from the TFL1 cDNA (Bradley et al., Science, 275:80-83 (1997)) using primer JH1067 (SEQ ID NO: 11), a chimeric primer whose 5′ end consisted of FT sequence, and JH1075 (SEQ ID NO: 19). The resulting product consisted mostly of a TFL1 fragment linked to a short FT sequence at the 5′ end. The products were gel-purified and saved for the second round PCR. The final chimera, pJA1058, was generated by PCR using all three fragments generated in the first round of PCR and two primers, JH1089 (SEQ ID NO: 21), an FT primer with a KpnI-restriction site at its 5′ end, and JH1090 (SEQ ID NO: 22), an FT primer with a XbaI site (FIG. 4C). The resulting product was cloned and verified by sequencing. The primer sequences are given below. JH1091 (SEQ ID NO: 23) and JH1092 (SEQ ID NO: 24) are TFL1 primers with KpnI and XbaI sites added, respectively.

### EXAMPLE 2

### Generation of the Chimeric Genes by Swapping Regions within an Exon

The above method can be used to produce PEBP chimeras with swapped segments within an exon. The second round of fourth-exon chimeras was generated in a similar manner to the above example, except that the shorter fragments were not generated by PCR, but by directly annealing the synthetic complementary oligonucleotides. Primer sequences used to generate the various fourth exon chimeras (for example, pJA1120 through pJA1132 in FIG. 2) are given below.

| | | |
|---|---|---|
| JH1125 | CTCCCTCTGACAATTGTAGAAAACTG | (SEQ ID NO:25) |
| JH1126 | TTCTCTTTGTGCGTTAAAGAAGACG | (SEQ ID NO:26) |
| JH1127 | GTTCTGGCGCCACCCTGGT | (SEQ ID NO:27) |
| JH1128 | GTGATCTCTCGAAGGGATATTAGGAA | (SEQ ID NO:28) |
| JH1129 | CTGTCGAAACAATATAAACACGACAC | (SEQ ID NO:29) |
| JH1130 | CTGCCTGAACAGAACAAACACAAAC | (SEQ ID NO:30) |
| JH1131 | GTGTCGTGTTTATATTGTTTCGACAGA | (SEQ ID NO:31) |
| | AGCAAAGACGTGTTATCTTTCCT | |
| JH1132 | GTTTGTGTTTGTTCTGTTCAGGCAGCT | (SEQ ID NO:32) |
| | TGGCAGGCAAACAGTGTA | |
| JH1133 | ACCAGGGTGGCGCCAGAACTTCAACAC | (SEQ ID NO:33) |
| | TCGTAAATTTGCGGT | |
| JH1134 | TTCCTAATATCCCTTCGAGAGATCACT | (SEQ ID NO:34) |
| | TCAACACTCGCGAGTTTGCT | |
| JH1135 | CTTGGCAGGCAAACAGTGTATGCACCA | (SEQ ID NO:35) |
| | GGGTGGCGCCAGAAC | |
| JH1136 | GTTCTGGCGCCACCCTGGTGCATACAC | (SEQ ID NO:36) |
| | TGTTTGCCTGCCAAG | |
| JH1137 | AAGCAAAGACGTGTTATCTTTCCTAAT | (SEQ ID NO:37) |
| ATCCCTTCGAGAGATCAC | | |
| JH1138 | GTGATCTCTCGAAGGGATATTAGGAAA | (SEQ ID NO:38) |
| | GATAACACGTCTTTGCTT | |

The generation of PEBP chimeras with swapped segments within an exon is demonstrated in the following example, wherein the chimera pJA1122 is prepared. To generate pJA1122, a fragment was amplified from the FT cDNA (Kardailsky, I., supra) using primers JH1134 (SEQ ID NO: 34) and JH1065 (SEQ ID NO: 9). Another fragment was generated from the same cDNA by using primers JH1061 (SEQ ID NO: 5) and JH 1129 (SEQ ID NO: 29). The products were gel-purified and saved for the second round PCR. Oligonucleotides JH 1137 (SEQ ID NO: 37) and JH 1138 (SEQ ID NO: 38) were synthesized to generate a fragment corresponding to part of the TFL1 cDNA (Bradley, D. J., supra). The final chimera, pJA1122, was generated by amplifying:

1) the two fragments obtained from the first round (described in the above paragraph),

2) combined with JH1131 (SEQ ID NO: 31), JH1137 (SEQ ID NO: 37), and JH1138 (SEQ ID NO: 38),

3) combined with the primers JH1089 (SEQ ID NO: 21) and JH1090 (SEQ ID NO: 22), to add restriction sites KpnI and XbaI to the 5′ and 3′ ends (described previously).

The final chimera encodes a protein with the sequence listed in SEQ ID NO: 41.

### EXAMPLE 3

### Transformation of The Chimeric Genes To Arabidopsis

For plant transformation of the chimeric genes, the plasmid pCHF3, a modified version of pPZP212 (Hajdukiewicz et al., Plant Mol. Biol., 25:989-994 (1994)) was used. The vector was modified to contain a cauliflower mosaic virus 35S promoter (Odell et al., Nature, 313:810-812 (1985)) upstream of the multiple cloning site. The vector also contained a transcriptional terminator region derived from the gene encoding the small subunit of ribulose-1,5-bisphosphate carboxylase (rbcS). The chimeric sequences were excised from the pBluescript or pGEM vectors at the KpnI and XbaI sites, and subsequently subcloned into the same sites in the pCEF3 vector.

Following this method, several plasmid vectors (labeled pJA1055 to pJA1 132) were prepared (FIG. 2). Plasmids from pJA1055 to pJA1069 represent exon-swap chimeric genes, while plasmids pJA1120 to pJA1132 denote domain-swap chimeric genes. The transformation vectors were mobilized into Agrobacterium tumefaciens strain ASE (Fraley et al., Biotechnology, 3:629-635 (1985)). The constructs were introduced into ft-1 tfl1-1double mutants (Columbia background) by vacuum infiltration (Bechtold and Pelletier, Methods Mol Biol., 82:259-266 (1998)). Transgenic plants were selected on MS medium plates containing 50 mg/L kanamycin. After growing T1 seedlings on MS plates for 10 days under long day conditions (16 h light; 8 h dark), at least 12 transgenic seedlings for each construct were transferred to soil and grown under long day conditions. As a negative control, ft-1 tfl1-1 seedlings grown under the same condition without kanamycin addition were transferred to soil.

Flowering time was measured by counting primary rosette leaves and cauline leaves when the plants were at the bolting stage of growth, prior to flowering. Results of the flowering time measurements for the various chimeras are shown below in Tables 1 and 2. The number of leaves produced before flowers are produced is indicated. In comparison, wild-type plants growing under these conditions produce approximately 12-14 leaves before flowering. Table 1 shows the number of leaves before flowering in transgenic plants containing swaps of entire exons. The experiment was performed using 14 lines and three separate controls.

TABLE 1

| | Range* | Average | SD | n |
|---|---|---|---|---|
| pJA1055 | 29-48 | 39.2 | 7.0 | 11 |
| pJA1056 | 7-33 | 17.1 | 9.5 | 11 |
| pJA1057 | 21-49 | 38.8 | 8.1 | 12 |
| pJA1058 | 15-20 | 17.5 | 1.9 | 12 |
| pJA1059 | 25-53 | 41.1 | 6.8 | 12 |
| pJA1060 | 11-18 | 13.4 | 2.1 | 12 |
| pJA1061 | 42-57 | 47.8 | 4.0 | 12 |
| pJA1063 | 16-31 | 24.7 | 5.0 | 12 |
| pJA1064 | 23-42 | 30.4 | 5.2 | 11 |
| pJA1065 | 10-20 | 14.3 | 3.3 | 12 |
| pJA1066 | 35-51 | 42.8 | 4.5 | 12 |
| pJA1067 | 20-41 | 31.2 | 5.2 | 12 |
| pJA1068 | 23-41 | 32.1 | 5.4 | 12 |
| pJA1069 | 26-31 | 28.6 | 1.6 | 12 |
| ft-1 tfl1-1 #1 | 26-32 | 28.4 | 1.6 | 11 |
| ft-1 tfl1-1 #2 | 25-36 | 29.4 | 3.0 | 12 |
| ft-1 tfl1-1 #3 | 26-30 | 28.0 | 1.5 | 12 |

SD standard deviation
*number of rosette leaves
n number of plants

Table 2 shows the number of leaves before flowering in transgenic plants containing swaps of segments within the 4^{th} exon. This experiment was performed with 13 lines and one control.

TABLE 2

| | Range* | Average | SD | n |
|---|---|---|---|---|
| pJA1120 | 25-52 | 41.2 | 6.1 | 18 |
| pJA1121 | 20-43 | 32.6 | 4.5 | 18 |
| pJA1122 | 44-77 | 52.6 | 9.0 | 18 |
| pJA1123 | 25-47 | 35.3 | 5.9 | 18 |
| pJA1124 | 30-56 | 45.3 | 7.5 | 18 |
| pJA1125 | 25-48 | 37.8 | 7.4 | 17 |
| pJA1126 | 2-14 | 4.9 | 2.1 | 17 |
| pJA1127 | 2-6 | 4.3 | 0.8 | 18 |
| pJA1128 | 34-47 | 41.2 | 3.3 | 18 |
| pJA1129 | 25-49 | 37.6 | 7.4 | 18 |
| pJA1130 | 29-62 | 50.6 | 6.8 | 17 |
| pJA1131 | 43-56 | 49.2 | 5.0 | 5 |
| pJA1132 | 30-58 | 49.7 | 7.7 | 18 |
| ft-1 tf1-1 #3 | 19-27 | 22.9 | 2.2 | 17 |

SD standard deviation
*number of rosette leaves
n number of plants

### EXAMPLE 4

### Transformation Of Tobacco And Other Plant Species

For tobacco transformation with the chimeric PEBP genes, genes were subcloned into the pCHF3 vector (Hajdukiewicz et al., supra (1994)) at the KpnI and XbaI sites as described in example 1, above. The final constructs thus contained a CaMV 35S promoter and an rbcS transcriptional terminator. The vectors were then transformed to tobacco leaf disks by Agrobacterium-mediated transformation following the method of Horsch et al. (Science, 227:1229-1231 (1985)).

The 35S::FT was previously transformed into tobacco, dramatically accelerating flowering (cited in FT patent application). Transformation of tobacco with 35S::TFL1 and 35S::CEN has been reported by others (Amaya et al., Plant Cell, 11:1405-1418 (1999)).

### EXAMPLE 5

### Method For Identifying A Compound That Affects The Activity or Expression of a Chimeric Early PEBP/Late PEBP Encoding Gene

Seeds derived from the Arabidopsis plants transformed as in example 2 with the chimeric PEBP-encoding constructs are germinated and grown for 3 weeks in sterile soil supplemented with 0.5×Hoagland's Solution at 25° C. with a 16 hour photoperiod. The test compounds are diluted to several concentrations and sprayed on the leaf surface on a biweekly basis. Control plants are sprayed with an equal volume of H₂O only. Compounds that alter activity of the chimeric PEBP protein may be identified by their effect on flowering time by measuring the number of leaves produced before the onset of flowering. Plants with modulated flowering characteristics may then be examined for alterations in levels of the chimeric PEBP protein by an immunoblot using an antibody to the chimeric gene product. Additionally, changes in expression levels of the chimeric PEBP gene may be measured by northern blot analysis using probes specific to the chimeric mRNA by the following method. RNA is isolated from the treated Arabidopsis plants. The RNA is fractionated in formaldehyde denaturing agarose gels, transferred to nitrocellulose filters, and hybridized with ³²P-labeled probes specific to the chimeric mRNA. The filters are then washed in 2×SSC, 1% SDS for 30 minutes at 70° C., followed by several 5 minute washes in 1×SSC, 1% SDS (Sambrook et al., 1989 Molecular Cloning: A Laboratory Manual. Cold Spring Harbor Laboratory Press, Cold Spring Harbor, N.Y. (1989)).

Conclusion

All references cited throughout the specification are hereby expressely incorporated by reference.

While the present invention has been described with reference to the specific embodiments thereof, it would be understood by those skilled in the art that various changes may be made and equivalents may be substituted without departing from the true spirit and scope of the invention. In addition, many modifications may be made to adapt a particular situation, material, composition of matter, process, process step or steps, to the objection, spirit and scope of the present invention. All such modifications are intended to be within the scope of the claims appended hereto.

What is claimed is:

1. A chimeric polypeptide comprising at least one region of an early-flowering phosphatidylethanolamine-binding protein, combined with at least one region of a late-flowering PEBP polypeptide, wherein the chimeric polypeptide has a different flowering time from that of a wild-type PEBP polypeptide.

2. The polypeptide according to claim 1, wherein the at least one region is substantially the same as a region of SEQ ID NO: 3.

3. The polypeptide according to claim 1, wherein the at least one region is substantially the same as a region of SEQ ID NO: 4.

4. The polypeptide according to claim 1, wherein the amino acid sequence is set forth in SEQ ID NO: 6.

5. The polypeptide according to claim 1, wherein the region transferred from TFL1 comprises about amino acids #131-145 of SEQ ID NO: 4.

6. The polypeptide according to claim 1 wherein the amino acid sequence is set forth in SEQ ID NO: 8.

7. The polypeptide according to claim 1, wherein the region transferred from TFL1 comprises about amino acids #146-171 of SEQ ID NO: 4.

8. An isolated polynucleotide encoding the polypeptide of claim 1.

9. An isolated polynucleotide selected from the group consisting of:
a) At least one region of SEQ ID NO: 1, or a sequence having substantial similarity thereto, combined with at least one region of SEQ ID NO: 2, or a sequence having substantial similarity thereto; and
b) At least one region of SEQ ID NO: 1, or a sequence having substantial similarity thereto, combined with at least one region of SEQ ID NO: 2, or a sequence having substantial similarity thereto, wherein T can also be U.

10. An isolated polynucleotide according to claim 9 wherein the nucleotide sequence is set forth in SEQ ID NO: 5.

11. The polynucleotide of claim 9, wherein the polynucleotide is isolated from a plant cell.

12. A recombinant expression vector comprising a polynucleotide sequence according to claim 9.

13. A host cell containing the vector of claim 9.

14. An antibody which binds to the polypeptide of claim 1, or binds to antigenic fragments of said polypeptide.

15. A genetically modified plant comprising at least one exogenous nucleic acid sequence comprising at least one region from an early PEBP-encoding nucleic acid sequence plus at least one region from a late PEBP-encoding nucleic acid sequence in its genome.

16. A plant of claim 15, further characterized as having modulated flower development.

17. The plant of claim 16, wherein the modulation is accelerated flower development.

18. The plant of claim 16, wherein the modulation is inhibited flower development.

19. The plant of claim 15, further comprising an exogenous gene encoding a polypeptide selected from the group consisting of LEAFY (LFY), APETALA1 (AP1), CONSTANS (CO), TERMINAL FLOWER1 (TFL1), FLORICAULA (FLO), FLOWERING LOCUS T (FT), SQUAMOSA (SQUA), FLOWERING LOCUS CA (FCA) and combinations thereof.

20. The plant of claim 15, wherein the exogenous gene is operably associated with a regulatory nucleic acid sequence.

21. The plant of claim 20, wherein the regulatory nucleic acid sequence is a promoter.

22. The plant of claim 21, wherein the promoter is a constitutive promoter.

23. The plant of claim 21, wherein the promoter is an inducible promoter.

24. The plant of claim 21, wherein the promoter is a tissue-specific promoter.

25. The plant of claim 15, wherein the nucleic acid further comprises a selectable marker.

26. The plant of claim 15, wherein the plant is a dicotyledonous plant.

27. The plant of claim 15, wherein the plant is a monocotyledonous plant.

28. A plant cell derived from the plant of claim 15.

29. Plant tissue derived from the plant of claim 15.

30. A seed which germinates into a plant comprising at least one exogenous nucleic acid sequence comprising at least one region of an early PEBP-encoding nucleic acid sequence plus at least one region of a late PEBP-encoding nucleic acid sequence in its genome and characterized as having modulated flower development.

31. A vector containing a nucleic acid sequence of claim 1.

32. The vector of claim 31, wherein the vector comprises a T-DNA derived vector.

33. The vector of claim 31, wherein the structural gene encodes a chimeric polypeptide comprising at least one region of an early PEBP polypeptide combined with at least one region of a late PEBP polypeptide.

34. The vector of claim 33, further comprising a structural gene encoding a polypeptide selected from the group consisting of LEAFY (LFY), APETALA1 (AP1), CONSTANS (CO), TERMINAL FLOWER1 (TFL1), FLORICAULA (FLO), FLOWERING LOCUS T (FT), SQUAMOSA (SQUA), FLOWERING LOCUS CA (FCA) and combinations thereof.

35. The vector of claim 33, wherein the promoter is a constitutive promoter.

36. The vector of claim 33, wherein the promoter is an inducible promoter.

37. The vector of claim 33, wherein the promoter is induced by chemical means.

38. The vector of claim 33, wherein the promoter is a tissue-specific promoter.

39. A method for genetically modifying a plant cell such that a plant, produced from said cell, is characterized as having modulated flower development as compared with a wild-type plant, said method comprising:
a) introducing at least the chimeric early PEBP/late PEBP-encoding polynucleotide of claim 1 into a plant cell to obtain a transformed plant cell; and
b) growing the transformed plant cell under conditions which permit expression of the early PEBP/late PEBP chimeric polypeptide, thereby producing a plant having modulated flower development.

40. The method of claim 39, wherein said modulation is accelerated flower development.

41. The method of claim 39, wherein the modulation is inhibited flower development.

42. The method of claim 39, wherein said accelerated flower development is achieved by inducing expression or activity of the chimeric early PEBP/late PEBP in the plant.

43. The method of claim 39, wherein said accelerated flower development is achieved by augmenting expression or activity of the early PEBP/late PEBP chimera in the plant.

44. The method of claim 41, further comprising inducing expression or activity of the Leafy (LFY) gene.

45. A method of producing a genetically modified plant characterized as having early flower development, said method comprising:
contacting a plant cell with a vector containing a nucleic acid sequence comprising at least a structural gene encoding the chimeric early PEBP/late PEBP polypeptide, said gene operably associated with a promoter, to obtain a transformed plant cell;
producing a plant from said transformed plant cells; and
selecting a plant exhibiting said early flower development.

46. The method of claim 45, wherein the contacting is by physical means.

47. The method of claim 45, wherein the contacting is by chemical means.

48. The method of claim 45, wherein the plant cell is selected from the group consisting of protoplasts, gamete producing cells, and cells which regenerate into a whole plant.

49. The method of claim 45, wherein the promoter is a constitutive promoter.

50. The method of claim 45, wherein the promoter is an inducible promoter.

51. A plant produced by the method of claim 45.

52. Plant tissue derived from a plant produced by the method of claim 45.

53. A method for modulating flower development in a plant cell comprising:
contacting plant cell with the vector of claim 33 to obtain a transformed plant cell;
growing the transformed plant cell under plant forming conditions; and
inducing early flower development in the plant under conditions and for a time sufficient to accelerate flower development.

54. A method for identifying a compound that affects activity or expression of an early PEBP/late PEBP chimera comprising:
a) Incubating components comprising the compound and the chimeric early PEBP/late PEBP polypeptide or a recombinant cell expressing the early PEBP/late PEBP chimeric polypeptide, under conditions that allow the components to interact; and
b) Determining the effect of the compound on chimeric early PEBP/late PEBP activity or expression.

55. The method of claim 54, wherein the effect is inhibition of chimeric early PEBP/late PEBP activity or expression.

56. The method of claim 54, wherein the effect is stimulation of chimeric early PEBP/late PEBP activity or expression.


SEQUENCE LISTING (41 sequences)

SEQ ID NO: 1 - DNA, 831 bp, ARABIDOPSIS THALIANA
acaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttat

SEQ ID NO: 2 - DNA, 668 bp, ARABIDOPSIS THALIANA
agttaacaaaagaaaatggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctagtttcatgattgtcataaactgcaaaaatgaaagaagaaaatttgcatgtaatctcatgtttatttgtgttctgaatttccgtactctgaataaaaactgccaaagatgagttgaatccg

SEQ ID NO: 3 - protein, 175 aa, ARABIDOPSIS THALIANA
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQLGRQTVYAPGWRQNFNTREFAEIYNLGLPVAAVFYNCQRESGCGGRRL

SEQ ID NO: 4 - protein, 136 aa, ARABIDOPSIS THALIANA
MENMGTRVIEPLIMGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQTAARKR

SEQ ID NO: 5 - DNA, 34 bp, ARABIDOPSIS THALIANA
atgtctataaatataagagaccctcttatagtaa

SEQ ID NO: 6 - DNA, 26 bp, ARABIDOPSIS THALIANA
caaagtatagaagttcctgaggtctt

SEQ ID NO: 7 - DNA, 22 bp, ARABIDOPSIS THALIANA
caatggagatattctcggaggt

SEQ ID NO: 8 - DNA, 23 bp, ARABIDOPSIS THALIANA
caaaggttgttccagttgtagca

SEQ ID NO: 9 - DNA, 21 bp, ARABIDOPSIS THALIANA
ctaaagtcttcttcctccgca

SEQ ID NO: 10 - DNA, 22 bp, ARABIDOPSIS THALIANA
atggagaatatgggaactagag

SEQ ID NO: 11 - DNA, 44 bp, ARABIDOPSIS THALIANA
cctcaggaacttctatactttggtgatgatagacccagatgttc

SEQ ID NO: 12 - DNA, 45 bp, ARABIDOPSIS THALIANA
tcacctccgagaatatctccattggatcgttacaaacattcccgg

SEQ ID NO: 13 - DNA, 44 bp, ARABIDOPSIS THALIANA
tgctacaactggaacaacctttggcaaagaggtggtgagctatg

SEQ ID NO: 14 - DNA, 18 bp, ARABIDOPSIS THALIANA
ctagcgtttgcgtgcagc

SEQ ID NO: 15 - DNA, 46 bp, ARABIDOPSIS THALIANA
gatctcagatccttcttcactttggttatggtggatccagatgttc

SEQ ID NO: 16 - DNA, 45 bp, ARABIDOPSIS THALIANA
ctttctaaaagaacacctgcactggttggtgactgatatccctgc

SEQ ID NO: 17 - DNA, 44 bp, ARABIDOPSIS THALIANA
gcacaacagatgctacgtttggcaatgagattgtgtgttacgaa

SEQ ID NO: 18 - DNA, 24 bp, ARABIDOPSIS THALIANA
caaagtgaagaaggatctgagatc

SEQ ID NO: 19 - DNA, 24 bp, ARABIDOPSIS THALIANA
cagtgcaggtgttcttttagaaag

SEQ ID NO: 20 - DNA, 21 bp, ARABIDOPSIS THALIANA
caaacgtagcatctgttgtgc

SEQ ID NO: 21 - DNA, 42 bp, ARABIDOPSIS THALIANA
gaggtaccatgtctataaatataagagaccctcttatagtaa

SEQ ID NO: 22 - DNA, 29 bp, ARABIDOPSIS THALIANA
gatctagactaaagtcttcttcctccgca

SEQ ID NO: 23 - DNA, 30 bp, ARABIDOPSIS THALIANA
aaggtaccatggagaatatgggaactagag

SEQ ID NO: 24 - DNA, 26 bp, ARABIDOPSIS THALIANA
tctctagactagcgtttgcgtgcagc

SEQ ID NO: 25 - DNA, 26 bp, ARABIDOPSIS THALIANA
ctccctctgacaattgtagaaaactg

SEQ ID NO: 26 - DNA, 25 bp, ARABIDOPSIS THALIANA
ttctctttgtgcgttaaagaagacg

SEQ ID NO: 27 - DNA, 19 bp, ARABIDOPSIS THALIANA
gttctggcgccaccctggt

SEQ ID NO: 28 - DNA, 26 bp, ARABIDOPSIS THALIANA
gtgatctctcgaagggatattaggaa

SEQ ID NO: 29 - DNA, 26 bp, ARABIDOPSIS THALIANA
ctgtcgaaacaatataaacacgacac

SEQ ID NO: 30 - DNA, 25 bp, ARABIDOPSIS THALIANA
ctgcctgaacagaacaaacacaaac

SEQ ID NO: 31 - DNA, 50 bp, ARABIDOPSIS THALIANA
gtgtcgtgtttatattgtttcgacagaagcaaagacgtgttatctttcct

SEQ ID NO: 32 - DNA, 45 bp, ARABIDOPSIS THALIANA
gtttgtgtttgttctgttcaggcagcttggcaggcaaacagtgta

SEQ ID NO: 33 - DNA, 42 bp, ARABIDOPSIS THALIANA
accagggtggcgccagaacttcaacactcgtaaatttgcggt

SEQ ID NO: 34 - DNA, 47 bp, ARABIDOPSIS THALIANA
ttcctaatatcccttcgagagatcacttcaacactcgcgagtttgct

SEQ ID NO: 35 - DNA, 42 bp, ARABIDOPSIS THALIANA
cttggcaggcaaacagtgtatgcaccagggtggcgccagaac

SEQ ID NO: 36 - DNA, 42 bp, ARABIDOPSIS THALIANA
gttctggcgccaccctggtgcatacactgtttgcctgccaag

SEQ ID NO: 37 - DNA, 45 bp, ARABIDOPSIS THALIANA
aagcaaagacgtgttatctttcctaatatcccttcgagagatcac

SEQ ID NO: 38 - DNA, 45 bp, ARABIDOPSIS THALIANA
gtgatctctcgaagggatattaggaaagataacacgtctttgctt

SEQ ID NO: 39 - protein, 15 aa, ARABIDOPSIS THALIANA
KQRRVIFPNIPSRDH

SEQ ID NO: 40 - protein, 26 aa, ARABIDOPSIS THALIANA
FNTRKFAVEYDLGLPVAAVFFNAQRE

SEQ ID NO: 41 - protein, 176 aa, Chimeric Amino Acid Sequence
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQKQRRVIFPNIPSRDHFNTREFAEIYNLGLPVAAVFYNCQRESGCGGRRL